(19) United States
(12) Patent Application Publication
(10) Pub. No.: US 2012/0265226 A1
(43) Pub. Date: Oct. 18, 2012

(54) Cotton fabric, nasal cleanser

(76) Inventor: Michael Barrington Walters, N. Miami, FL (US)

(21) Appl. No.: 12/932,888

(22) Filed: Apr. 14, 2011

(51) Int. Cl. A61B 17/24

(52) U.S. Cl. 606/162

(57) ABSTRACT

A nasal cleanser in the form of a glove for the pointing finger, made from a cotton/polyester blend of fabric that is white in color. The fabric is stitched or glued together by hand or machinery in the form of a glove for the finger with one open and one closed end. The closed end resembles the rounded shape of the finger tip. The glove is wider at the open end, making it easier for the user to insert the finger into the glove.

Described hereto is a new method for cleaning the inside of the nostrils, using a polyester/cotton blend fabric, in the form of a glove for the finger. This glove is white in color and is hand or machine stitched or glued, in the form of a cone or bell, making it wider at the open end, whereby making it easy for the user to insert the finger, and more narrow at the closed end, allowing for easy insertion into the nostrils. This product is then discarded after a single use, or is washable.

### BRIEF DESCRIPTION OF THE DRAWINGS

FIG. 1 Is a schematic raised or three dimensional view of a new Nasal Cleanser showing the opening where the forger is inserted.

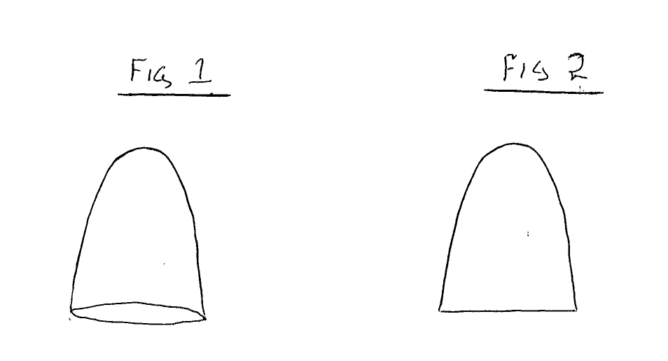

FIG. 2 Is a schematic flat or two dimensional view of a new Nasal Cleanser.

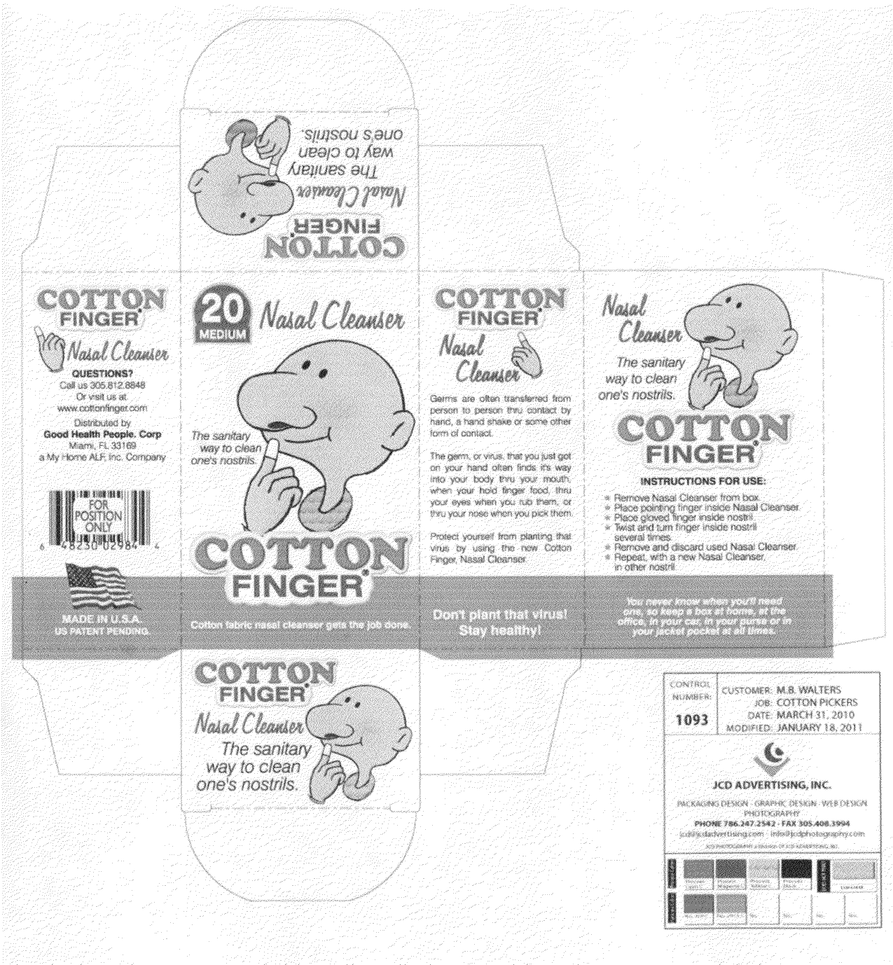

I claim:

1. A Nasal Cleanser, in the shape of a thimble, designed to fit the pointing finger.

2. A nasal cleanser made of a fabric blend of cotton and polyester.

3. A nasal cleanser white in color.

4. A nasal cleanser that is both disposable and washable.
